(19) United States
(12) Patent Application Publication
(10) Pub. No.: US 2010/0049283 A1
(43) Pub. Date: Feb. 25, 2010

(54) ILLUMINATION-BASED MEDICAL DEVICE & METHODS OF USE THEREOF

(75) Inventor: Randy E. Johnson, Escondido, CA (US)

Correspondence Address: Loza & Loza LLP, 305 North Second Ave., #127, Upland, CA, 91786, US

(73) Assignee: BLISS HOLDINGS, LLC, Escondido, CA (US)

(21) Appl. No.: 12/545,404

(22) Filed: Aug. 21, 2009

Related U.S. Application Data
(60) Provisional application No. 61/090,705, filed on Aug. 21, 2008.

(51) Int. Cl. A61N 5/067

(52) U.S. Cl. 607/89

(57) ABSTRACT

Embodiments of illumination-based medical devices capable of projecting a holographic symbol are herein provided. In one embodiment, an illumination-based medical device incorporates at least a laser and a diffractive optical element within a housing. In one embodiment, the diffractive optical element may be a holographic optical element. In a particular embodiment, the holographic optical element may incorporate an interference pattern and/or a sacred symbol or pattern.

### CLAIM OF PRIORITY

This application claims the benefit of U.S. Provisional Application No. 61/090,705 entitled “Illumination-Based Medical Device and Methods of Use Thereof,” filed on Aug. 21, 2008.

### FIELD OF INVENTION

Illumination-based medical devices.

### BACKGROUND OF INVENTION

Light may be considered as a fundamental force of life. Light provides energy and facilitates the metabolic processes essential to life. For example, scientific research has shown that diminished exposure to natural sunlight can negatively affect a person's mood. Artificial light sources play an increasing significant role in medicinal and aesthetic applications. Lasers are examples of the latest and the most advanced artificial light sources. They generate artificial light and amplify it into focused intense beams of light.

When laser light is absorbed by a living tissue, it triggers biological reactions in the cells. A variety of endogenous chemical substances are produced in the cells and carried by blood and lymphatic flow to other parts of the system. Hence, it is thought that the effects of cold laser light may not only be local, but can also achieve wide systemic effects.

The main and the most important effect of laser light on cells is thought to be the accelerated production of adenosine triphosphate (ATP). ATP molecules are found in the cells of all living things. In animal and human systems, ATP is synthesized in small cellular organelles called mitochondria. In the mitochondria, the primary cellular energy source, i.e., ATP, is produced by combining oxygen with sugar derived from food.

ATP can be described as the “energy carrier” or the “energy shuttle” capable of harnessing the chemical energy generated from the breakdown of the foodstuffs and transporting it across cellular membranes for conversion into “fuel” that is required for normal body functioning. ATP is often referred to as the “energy currency of life.”

If a person has insufficient levels of ATP available, the energy cannot reach the tissues. This can lead to a variety of health problems, such as a susceptibility to infectious diseases, poor wound healing, inflammation and swelling.

In a process termed phototherapy, a low level laser therapy device (soft lasers or cold lasers) can deliver light into living tissues thereby increasing ATP and shuttling more energy and nutrients around the body for healthy metabolism and the appropriate functioning of organs. Lasers exert photochemical effects rather than thermal effects typically effectuated by heat-lamp based artificial light sources. Lasers are consequently referred to as “cold” light sources.

Low level laser therapy devices (i.e., cold lasers and soft lasers) have been in medical and veterinary use for approximately 35 years. In the USA, the FDA recently approved therapeutic/medical low level lasers for human use because of their excellent safety with no significant risks attached.

### SUMMARY OF THE INVENTION

A medical device, comprising: (i) a cylindrical housing having an opening at a distal end; (ii) a laser housed within the housing and directed toward the opening; (iii) a holographic optical element within the housing, the holographic optical element positioned within a light pathway of the laser when power is supplied thereto; and (iv) a mechanism to switch the laser between an ON position and an OFF position is herein disclosed.

The holographic optical element may incorporate an interference pattern and optionally may incorporate a sacred symbol or a sacred pattern. The sacred symbol or the sacred pattern may be one of the Om symbol, the Flower of Life pattern, the Lotus symbol, the Gayatri Yantra symbol, the Yin-Yang symbol, the Spiral symbol, the Hand of Fatima symbol, the Tomoe symbol, the Luna Goddess symbol, the Celtic Triad symbol or the Feng Shui symbol. The holographic optical element may be in contact with a glass frit, the glass frit housed within the housing and positioned within the light pathway of the laser when power is supplied thereto. The laser may be one of a diode pumped solid state laser or a direct diode laser. The laser may emanate at a power of less than 5 milliwatts. The medical device may further include one of a current regulating or a photodiode forward feedback circuit board electrically connected to the laser. The laser may be powered by one of a battery or an external power source.

A method of holistic healing, comprising: directing a laser beam onto the skin of a person wherein the laser beam passes through a holographic optical element incorporating an interference pattern and optionally a sacred symbol or sacred pattern is herein disclosed.

In one embodiment, the laser beam may be directed continuously. Alternatively, the laser beam may be directed continuously for a predetermined time period. In another embodiment, the laser beam may be flashed repeatedly. Alternatively, the laser beam may be flashed repeatedly for a predetermined time period. The predetermined time period may be between about 15 seconds and about 15 minutes. The sacred symbol or the sacred pattern may be one of the Om symbol, the Flower of Life pattern, the Lotus symbol, the Gayatri Yantra symbol, the Yin-Yang symbol, the Spiral symbol, the Hand of Fatima symbol, the Tomoe symbol, the Luna Goddess symbol, the Celtic Triad symbol or the Feng Shui symbol.

### BRIEF DESCRIPTION OF THE DRAWINGS

FIG. 1 illustrates an optical path set-up to produce a hologram.

FIG. 2 illustrates an interference simulation pattern computed from a line drawing.

FIG. 3 illustrates a frensel planar interference pattern produced from perfect symmetry of reference and reflected coherence.

FIG. 4 illustrates a version of the Om sacred symbol.

FIG. 5 illustrates a version of the Flower of Life sacred pattern.

FIG. 6 illustrates a perspective view of a laser-based medical device according to an embodiment of the invention.

FIG. 7 illustrates an exploded view of a cap of the laser-based medical device of FIG. 6 according to an embodiment of the invention.

FIG. 8A illustrates a front view of the laser-based medical device of FIG. 6.

FIG. 8B illustrates a cross-sectional view of the laser-based medical device of FIG. 8A taken along lines A-A.

FIG. 9A is an image of a diffraction grating with multiple diffractive optics.

FIG. 9B is another image of a diffraction grating with multiple diffractive optics.

FIG. 9C is yet another image of a diffraction grating with multiple diffractive optics.

### DETAILED DESCRIPTION

Embodiments of illumination-based medical devices capable of projecting a holographic symbol are herein provided. In one embodiment, an illumination-based medical device incorporates at least a laser and a diffractive optical element within a housing. In one embodiment, the diffractive optical element may be a holographic optical element. In a particular embodiment, the holographic optical element may incorporate an interference pattern and/or a sacred symbol or pattern.

### Illumination Elements

A diffractive optical element (DOE) is a class of optics that operates on the principle of diffraction. Traditional optical elements use their shape to bend light. By contrast, diffractive optics break apart incoming waves of light into a large number of waves which then recombine to form completely new waves. DOEs can function as grating, lenses, aspheric or any other type of optical element. They offer unique optical properties that are not possible with conventional optical elements. In addition, DOEs can be fabricated in a wide range of materials including, but not limited to, aluminum, silicon, silica or plastic.

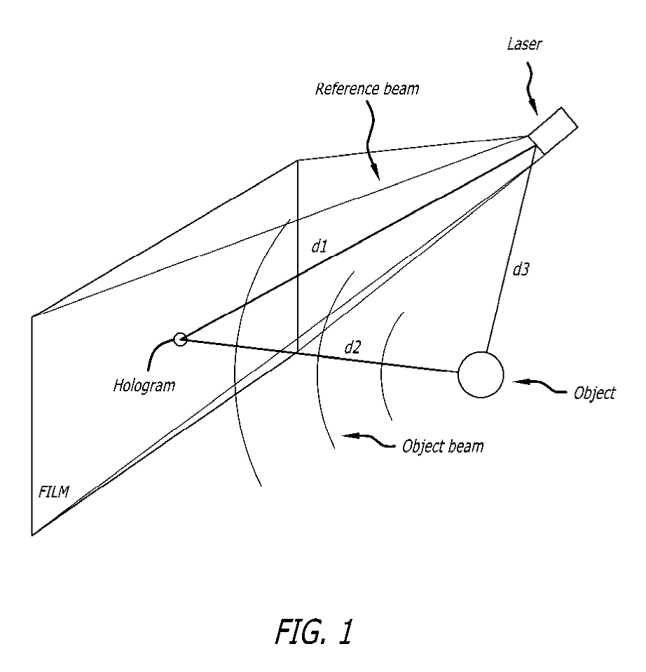

Holography is a technique that allows the light scattered from an object to be recorded and later reconstructed so that it appears as if the object is in the same position relative to the recording medium as it was when recorded. The image changes as the position and orientation of the viewing system changes in exactly the same way is if the object were still present, thus making the recorded image, termed a hologram, appear three-dimensional. FIG. 1 illustrates an optical path set-up to produce a hologram.

A hologram can be produced from laser-light beams being scattered off of an object and interfered with by a reference beam. A two-dimensional recording medium, such as a photosensitive plate or holographic film, records three-dimensional volumetric phase information of an object which is termed a fringe or iterative Fourier transfer algorithm (IFTA) pattern. This procedure is similar to photography where white light scattered from photographed objects is recorded on silver halide film. Light has a phase (volume) and amplitude (intensity) but only intensity is recorded in conventional photography. A hologram, however, stores both amplitude and phase due to the interference of the reference beam. This reference beam possesses the same characteristics as scattered light because of the action of the laser. The phase information is the most important factor in holography because it provides the depth cues to the eyes and allows for an image to appear in three dimensions.

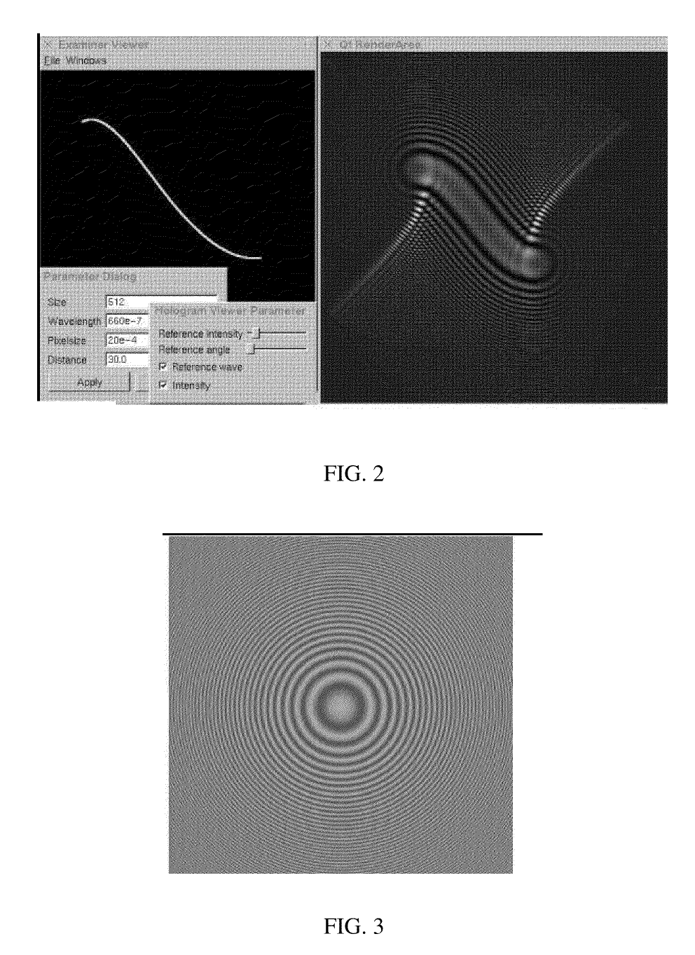

In the computer science field, a computer-generated holographic image is computed by numerically simulating the physical phenomena of light diffraction and interference. It is possible for computer software to calculate the phase of light reflected or transmitted from or through an object. Computing the phase of light of different objects, such as points, lines and wire frames, produces an interference simulation that may in turn by transferred to a photographically sensitive media. FIG. 2 illustrates an interference simulation pattern computed from a line drawing. FIG. 3 illustrates a frensel planar interference pattern produced from perfect symmetry of reference and reflected coherence (“Om” mantra).

A holographic optical element (HOE) is a type of DOE. A holographic optical element (HOE) is a hologram of a point source and acts as a lens or a mirror having optical power, i.e., the ability to focus light. The hologram consists of a diffraction pattern rendered as a surface relief which may be, for example, a thin film (created using photoresist and/or dichromated gelatin) containing an index modulation throughout the thickness of the film. “Index modulation” refers to a periodic feature set that has a linear distribution of patterns to produce novel optical effects created during the process of making the HOE. Either process (dichromated gelatin or photoresist) can be used to create a mathematical distribution to create a linear derivative producing a periodic feature set implemented into a phase mask. In one embodiment of the invention, a non-linear implementation of IFTA produces a logarithmic or otherwise hyperbolic IFTA wave function that may be used to produce non-linear phase derivatives onto a diffractive surface. According to embodiments of the invention, holograms can be classified into two categories: (i) “reflection holograms” in which incidence and diffracted light are on the same side of the HOE; and (ii) “transmission holograms” in which incident and diffracted light are on opposite sides.

### Sacred Symbols

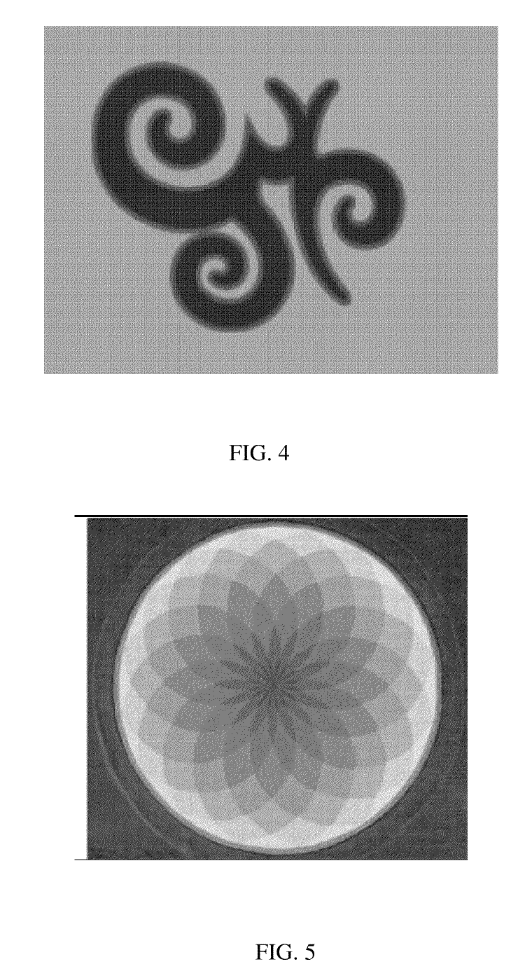

Many different cultures have sacred or mystical symbols or patterns incorporated within the culture. These symbols may have religious, philosophical and/or mythical connotations. Many believe that these symbols have healing powers as well. An example of a sacred symbol is the “Aum” (or “Om”), a mystical or sacred symbol in the Hindu, Jain and Buddhist religions. According to one interpretation, the Om sacred symbol represents both the unmanifest and manifest aspects of a monotheistic deity. FIG. 4 illustrates a version of the Om sacred symbol. An example of a sacred pattern is the Flower of Life, a sacred symbol used as a metaphor to illustrate the connectedness of all life and spirit within a universe. FIG. 5 illustrates a version of the Flower of Life sacred pattern. Other sacred symbols and/or patterns include, but are not limited to, the Lotus, the Gayatri Yantra, Yin-Yang, the Spiral, the Hand of Fatima, the Tomoe, the Luna Goddess, the Celtic Triad and the Feng Shui symbols; however, one of ordinary skill in the art will appreciate that many differing sacred symbols exist which may be incorporated as a healing modality.

### Illumination-Based Medical Devices

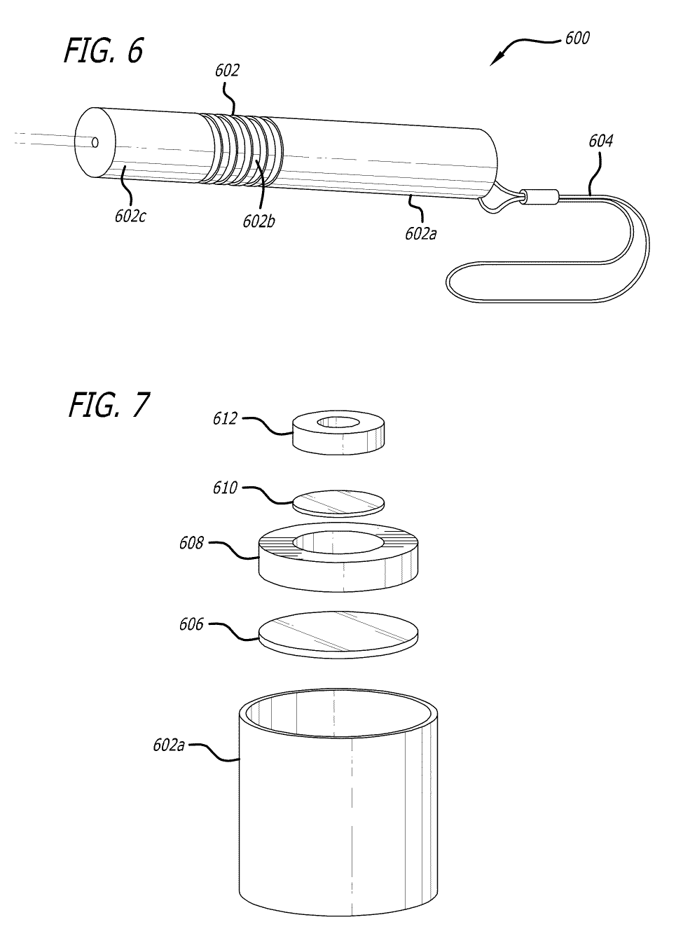

FIG. 6 illustrates a perspective view of a laser-based medical device according to an embodiment of the invention. As shown, laser-based medical device 600 includes a housing 602 which may house at least a laser (not shown) and a diffractive optical element (not shown). Housing 602 may be cylindrical in shape; however, other suitable geometric shapes are within the scope of the invention. Housing 602 may be made of a light-weight durable material such as aluminum or plastic. In some embodiments, laser-based medical device 600 may be from about three (3) inches to about six (6) inches in length, preferably about four (4) inches in length, and about 0.25 inches to about 3.5 inches in diameter, preferably about 0.75 inches in diameter. That is, laser-based medical device 600 may be capable of being held within a hand of a user.

Laser-based medical device 600 may include a proximal end 602a, a medial portion 602b and a distal end 602c (or “cap”, hereinafter referred to interchangeably). Proximal end 602a and medial portion 602b may be configured such that a user may hold the device 600 within his/her hand and distal end 602c may be adapted to threadedly engage with medial portion 602b. Laser-based medical device 600 may include an ON/OFF switch which may be any of those known by one of ordinary skill in the art. In one embodiment, the ON/OFF switch may be a push button ON/OFF switch located on an end-cap of proximal end 602a; however, other suitable ON/OFF mechanisms known by one of ordinary skill in the art are within the scope of the invention. According to some embodiments, the laser may be powered by a battery or by an external power source. A current regulating or photodiode forward feedback circuit board electrically connected to the laser, such as those known by one of ordinary skill in the art, may be used to regulate current flow to the laser. According to one embodiment, laser-based medical device 600 may include a wrist-securing mechanism 604.

In some embodiments, the laser of laser-based medical device 600 is a direct diode laser or a diode pumped solid state (DPSS) laser. A DPSS laser is a device that converts some form of energy (e.g., electrical, optical, chemical) into a narrow beam of light which is monochromatic, directional and coherent. “Monochromatic” means pertaining to light of one color or to radiation of a single wavelength or narrow range of wavelengths. “Directional” means that the beam of light is very well collimated and travels over long distances with very little spread in diameter. “Coherent” means of or pertaining to waves that maintain a fixed phase relationship. The effect of one wave enhances the strength of every other wave so that the overall effect of coherent light is much greater than if the waves were not in phase. In one embodiment, the DPSS laser may be an FLPPS Class 2 single beam laser with a total laser power of less than 5 milliwatts; however, higher power lasers may also be used.

FIG. 7 illustrates an exploded view of the cap 602c of laser-based medical device 600 of FIG. 6 according to an embodiment of the invention. In some embodiments, cap 602c may be internally threaded. Cap 602c may house a glass frit 606, a first washer 608, a diffractive optical element (DOE) 610 and a second washer 612, as shown. In one embodiment, DOE 610 is a holographic optical element (HOE) (hereinafter referred to interchangeably).

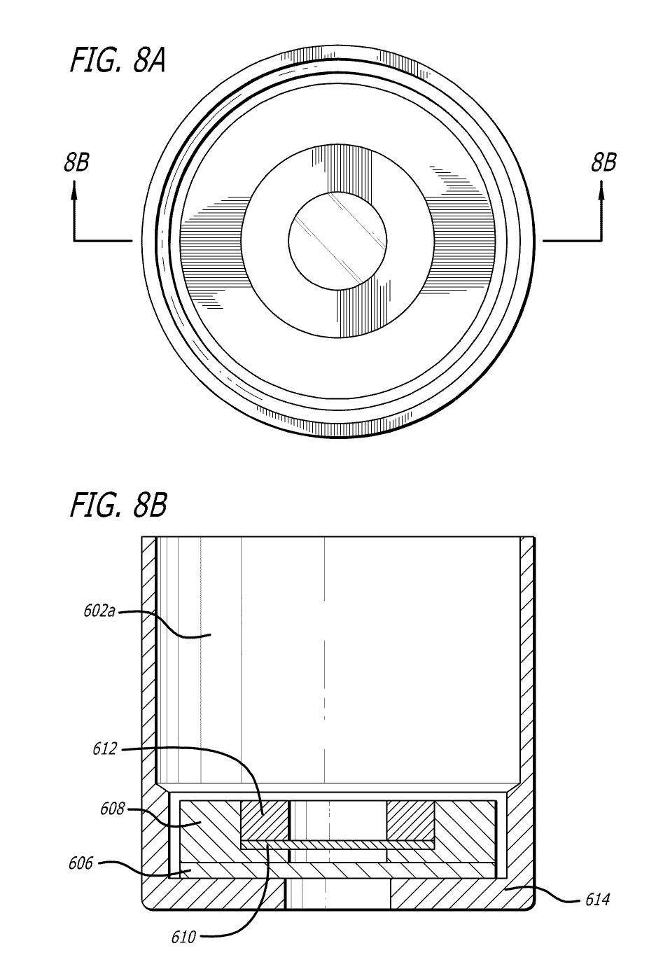

FIG. 8A illustrates a front view of the laser-based medical device of FIG. 6. FIG. 8B illustrates a cross-sectional view of the laser-based medical device of FIG. 8A taken along lines A-A. As illustrated, base cap 602c includes annular support 614 positioned at a base thereof, which annular support 614 functions to support glass frit 606. First washer 608 may be positioned above and adjacent to glass frit 606. In one embodiment, first washer 608 may be a shoulder washer or equivalent thereof. In some embodiments, glass frit 606 and first washer 608 may have the same or approximately the same circumference. HOE 610 may be positioned within an annular opening of first washer 608 and supported by washer annular support 616. In this manner, glass frit 606 remains at a distance from HOE 610. Second washer 612 may be positioned above and adjacent to HOE 610. In one embodiment, second washer 612 may be press-fit into first washer 608 to secure HOE 610. In some embodiments, HOE 610 and second washer 612 may have the same or approximately the same circumference.

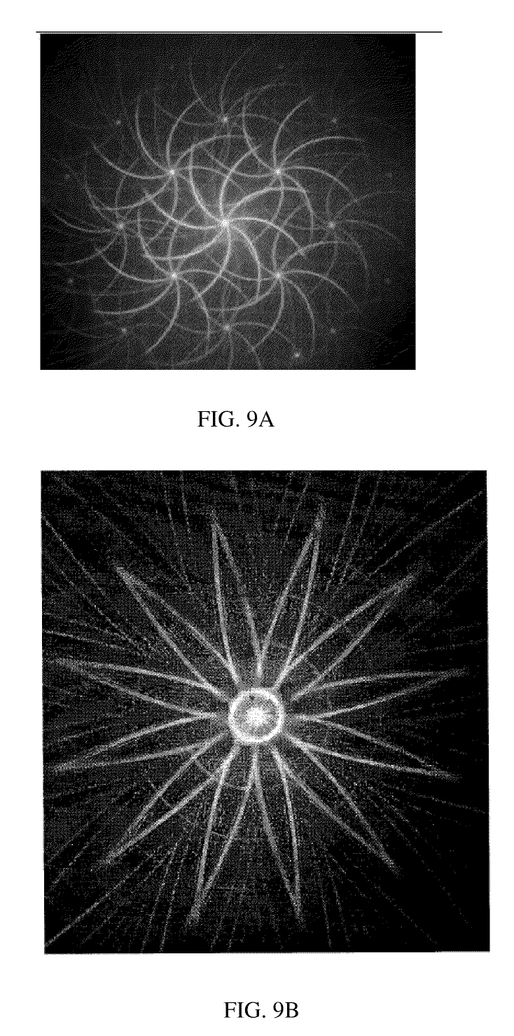

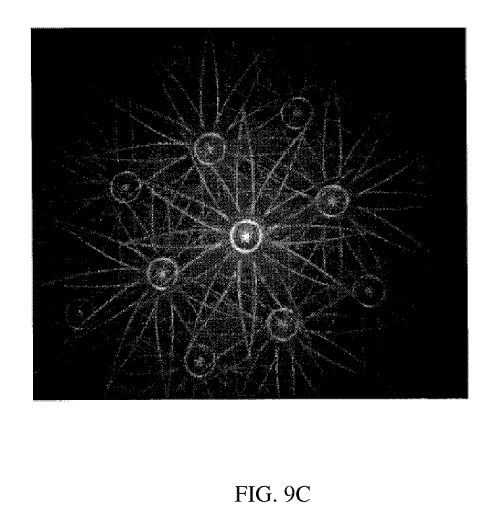

FIGS. 9A-9C are photographs of laser patterns formed by a laser-based medical device according to an embodiment of the invention. FIG. 9A is an image of a diffraction grating with multiple diffractive optics. FIG. 9B is another image of a diffraction grating with multiple diffractive optics. FIG. 9C is yet another image of a diffraction grating with multiple diffractive optics. According to some embodiments, the images may be created by situating a plurality of HOEs together within the housing or, alternatively, by recording of the specific image pattern onto the HOE itself. In one process, the HOE is created using lenticular grating. In another process, the HOE is created by using the multiplicity of layered diffraction gratings in any combination. More particularly, diffraction gratings are created by photoresist, i.e., the photoresist layer, then creation of a shim, then creation of replicates that are embossed on plastic or a polymer sheet. In yet another process, the HOE is created by using electron beam lithography. More particularly, a desired image or graphic is designed on a computer; then IFTA phase mask file (jpg) is calculated; then a master on a glass or fused silica quartz substrate is created through EB lithography; then the substrate is embossed.

In some embodiments, HOE 610 may include a hologram of a sacred symbol or sacred pattern. The symbol and/or pattern may be any of those described previously. Moreover, HOE 610 may be created by the method(s) described previously.

According to embodiments of methods of the invention, a laser-based medical device having an HOE incorporating a holographic sacred symbol or sacred pattern therein may be used as a holistic healing device. In one application, the laser light is projected onto the skin of a patient. The laser light projection may be continuous or repeatedly flashed for a predetermined time period and/or for a predetermined interval. In one embodiment, the predetermined time period is between about fifteen (15) seconds and about fifteen (15) minutes although longer treatment periods may be used. It is anticipated that the healing power from the sacred symbol and/or sacred geometric pattern passed on through the manner described (i.e., through the laser-based medical device) will be transferred to the patient. Also, it is anticipated that sacred symbol and/or sacred geometric pattern passed on through the manner described (i.e., through the laser-based medical device) will evoke and/or increase positive feelings (e.g., happiness, joy, pleasure) via a visual feedback loop which will cause the release of endorphins thereby contributing to the healing process. Advantageously, the laser-based medical device may provide healing both through the effects of phototherapy and through use of sacred symbols and/or sacred geometric patterns which may provide a synergistic healing effect.

While certain exemplary embodiments have been described and shown in the accompanying drawings, it is to be understood that such embodiments are merely illustrative of and not restrictive on the broad invention, and that this invention is not to be limited to the specific constructions and arrangements shown and described, since various other modifications may occur to those ordinarily skilled in the art.

What is claimed is:

1. A medical device, comprising:
a cylindrical housing having an opening at a distal end;
a laser housed within the housing and directed toward the opening;
at least one holographic optical element within the housing, the at least one holographic optical element positioned within a light pathway of the laser when power is supplied thereto; and
a mechanism to switch the laser between an ON position and an OFF position.

2. The medical device of claim 1 wherein the holographic optical element incorporates an interference pattern and optionally incorporates sacred symbol or a sacred pattern.

3. The medical device of claim 2 wherein the sacred symbol or the sacred pattern is one of the Om symbol, the Flower of Life pattern, the Lotus symbol, the Gayatri Yantra symbol, the Yin-Yang symbol, the Spiral symbol, the Hand of Fatima symbol, the Tomoe symbol, the Luna Goddess symbol, the Celtic Triad symbol or the Feng Shui symbol.

4. The medical device of claim 1 wherein the holographic optical element is in contact with a glass frit, the glass frit housed within the housing and positioned within the light pathway of the laser when power is supplied thereto.

5. The medical device of claim 1 wherein the laser is one of a diode pumped solid state laser or a direct diode laser.

6. The medical device of claim 1 wherein the laser emanates at a power of less than 5 milliwatts.

7. The medical device of claim 1, further comprising, one of a current regulating or a photodiode forward feedback circuit board electrically connected to the laser.

8. The medical device of claim wherein the laser is powered by one of a battery or an external power source.

9. A method of holistic healing, comprising:
directing a laser beam onto the skin of a person wherein the laser beam passes through a holographic optical element incorporating an interference pattern and optionally a sacred symbol or sacred pattern.

10. The method of claim 9 wherein the laser beam is directed continuously.

11. The method of claim 9 wherein the laser beam is directed continuously for a predetermined time period.

12. The method of claim 11 wherein the predetermined time period is between about 15 seconds and about 15 minute.

13. The method of claim 9 wherein the laser beam is flashed repeatedly.

14. The method of claim 9 wherein the laser beam is flashed repeatedly for a predetermined time period.

15. The method of claim 14 wherein the predetermined time period is between about 15 seconds and about 15 minutes.

16. The method of claim 9 wherein the sacred symbol or the sacred pattern is one of the Om symbol, the Flower of Life pattern, the Lotus symbol, the Gayatri Yantra symbol, the Yin-Yang symbol, the Spiral symbol, the Hand of Fatima symbol, the Tomoe symbol, the Luna Goddess symbol, the Celtic Triad symbol or the Feng Shui symbol.
